(19) United States
(12) Patent Application Publication
(10) Pub. No.: US 2018/0064483 A1
(43) Pub. Date: Mar. 8, 2018

(54) NON-SURGICAL ORBITAL FAT REDUCTION

(71) Applicant: Ronald L. Moy, Beverly Hills, CA (US)

(72) Inventor: Ronald L. Moy, Beverly Hills, CA (US)

(21) Appl. No.: 15/491,860

(22) Filed: Apr. 19, 2017

Related U.S. Application Data
(63) Continuation of application No. 14/022,752, filed on Sep. 10, 2013, now abandoned.
(60) Provisional application No. 61/699,090, filed on Sep. 10, 2012.

(51) Int. Cl. A61B 18/04; A61B 18/14

(52) U.S. Cl. CPC A61B 18/04; A61B 18/1485; A61B 2018/2005; A61F 2250/0001

(57) ABSTRACT

A method of nonsurgical orbital fat reduction includes providing an electromagnetic energy system that includes an electromagnetic energy source and a patient interface coupled to the electromagnetic energy source. The patient interface includes an elongate member configured to deliver electromagnetic energy generated by the electromagnetic energy source to tissue of a medical patient. The method also includes inserting at least a distal portion of the elongate member into an orbital fat pad of the medical patient and delivering a sufficient amount of electromagnetic energy from the electromagnetic energy source to the orbital fat pad to cause the orbital fat pad to shrink in volume.

### CROSS-REFERENCE TO RELATED APPLICATIONS

This application claims the benefit of priority from U.S. Provisional No. 61/699,090, filed Sep. 10, 2012, which is incorporated by reference in its entirety.

### FIELD

The technology relates generally to methods to reduce the volume of fat in a medical patient. In some embodiments, the technology relates to non-surgical methods of orbital fat reduction.

### BACKGROUND

The orbital septum is a membrane that extends from the orbital rims to the eyelids, and forms the fibrous portion of the eyelids. Excess skin, muscle and fat can cause an unattractive bulge to develop below the eye as the lower eyelid ages and the orbital septum weakens. Weakening of the orbital septum can lead to bulging of orbital fat and the appearance of puffiness around the eyes. Individuals with such conditions are sometimes referred to as having “bags” under their eyes.

Traditional techniques for correcting this condition involve surgical intervention. For example, blepharoplasty is a commonly performed aesthetic procedure to surgically remove the orbital fat around a patient's eyes. During blepharoplasty, an incision is made through the skin or lower eyelid conjunctiva to resect excessive orbital fat, redundant muscle, and skin, as needed.

However, such procedures are not without risk of complications. Indeed, postoperative changes in the shape of the aperture, inferior scleral show, and malposition of the lower eyelid may result. Therefore, a system and procedure for non-surgical orbital fat reduction is needed to address this condition while avoiding the risks associated with traditional surgical techniques.

### SUMMARY

The technology, in various aspects and embodiments, includes compositions, systems, and methods for non-surgical fat reduction. In some embodiments, the technology involves percutaneous delivery of electromagnetic energy to a fat to heat and shrink the fat. For example, the technology includes delivering one or more of radiofrequency (RF) energy, heat, electrical energy, optical energy (e.g., laser light, intense pulsed light, etc.), etc. to fat, such as an orbital fat pad, etc.

In one embodiment, a method of nonsurgical orbital fat reduction includes: providing an electromagnetic energy system, the electromagnetic energy system comprising an electromagnetic energy source and a patient interface coupled to the electromagnetic energy source, the patient interface comprising an elongate member configured to deliver electromagnetic energy generated by the electromagnetic energy source to tissue of a medical patient; inserting at least a distal portion of the elongate member into an orbital fat pad of the medical patient; and delivering a sufficient amount of electromagnetic energy from the electromagnetic energy source to the orbital fat pad to cause the orbital fat pad to shrink in volume.

In one embodiment, the orbital fat pad is selected from the group consisting of one or more of: a preaponeurotic fat pad, a retroseptal fat pad, a central fat pad, a medial fat pad, a lateral fat pad, and a brow fat pad. The method can also include orienting the elongate member such that the electromagnetic energy is not directed toward or delivered directly into tissue selected from the group consisting of one or more of: an eyeball, a trochlea, a lacrimal gland, a tendon and a muscle. The method can include orienting the elongate member such that the electromagnetic energy is not substantially absorbed by tissue near the fat pad selected from the group consisting of one or more of: an eyeball, a trochlea, a lacrimal gland, a tendon and a muscle. The electromagnetic energy source can be selected from the group consisting of one or more of: a radiofrequency generator, a laser, an intense pulsed light source, a thermal energy source, and an electrical energy source.

The method can also include selecting at least one of a frequency, energy level, power level, waveform, and duty cycle of the electromagnetic energy's waveform. The frequency can be selected to be about 1, 1.4, 1.7, 2, 3, 4, 5 MHz, or between 1 and 2 MHz. The power level can be selected to be about 20, 30, 50, 100, 150, or 200 W.

The method can also include adjusting a position of a stop coupled to the elongate member to control or limit a depth of insertion of the elongate member into the medical patient's orbital fat pad. The method can also include removing the elongate member from the orbital fat pad, inserting the elongate member into a different portion of the orbital fat pad, and delivering addition electromagnetic energy to the fat pad.

In some embodiments, inserting at least the distal end of the elongate member includes inserting at least the distal end of the elongate member through the medical patient's skin, inserting at least the distal end of the elongate member through the medical patient's conjunctiva, or inserting at least the distal end of the elongate member into the fat pad without penetrating the medical patient's skin.

Another embodiment of a method of nonsurgical orbital fat reduction includes: penetrating a medical patient's tissue with a distal end of a RF probe; advancing the distal end of the RF probe into an orbital fat pad; and delivering RF energy from the RF probe into the orbital fat pad to shrink the orbital fat pad volume.

The orbital fat pad may be selected from the group consisting of one or more of: a preaponeurotic fat pad, a retroseptal fat pad, a central fat pad, a medial fat pad, a lateral fat pad, and a brow fat pad. The method may also include orienting the distal end of the elongate probe such that the electromagnetic energy is not directed toward or delivered directly into tissue selected from the group consisting of one or more of: an eyeball, a trochlea, a lacrimal gland, a tendon and a muscle. The method may also include orienting the distal end of the elongate probe such that the electromagnetic energy is not substantially absorbed by tissue near the fat pad selected from the group consisting of one or more of: an eyeball, a trochlea, a lacrimal gland, a tendon and a muscle.

The method may also include selecting at least one of a frequency, energy level, power level, waveform, and duty cycle of the RF energy waveform. The frequency may be selected to be about 1, 1.4, 1.7, 2, 3, 4, 5 MHz, or between 1 and 2 MHz. The power level may be selected to be about 20, 30, 50, 100, 150, or 200 W.

In some embodiments, the method also includes adjusting a position of a stop coupled to the RF probe to control or limit a depth of insertion of the RF probe into the medical patient's orbital fat pad. The method may also include removing the distal end of the RF probe from the orbital fat pad, inserting the distal end of the RF probe into a different portion of the orbital fat pad, and delivering addition RF energy to the fat pad. In some embodiments, advancing the distal end of the RF probe includes inserting at least the distal end of the RF probe through the medical patient's skin, inserting at least the distal end of the RF probe through the medical patient's conjunctiva, or inserting at least the distal end of the RF probe into the fat pad without penetrating the medical patient's skin.

The various embodiments described herein can be complimentary and can be combined or used together.

### BRIEF DESCRIPTION OF FIGURES

FIG. 1 is a block diagram of a system configured to deliver electromagnetic energy to reduce the volume of an orbital fat pad in a medical patient;

FIG. 2 is a perspective view of the patient interface portion of the system of FIG. 1;

FIG. 3 is a partial cross sectional view of an embodiment of a tip compatible with the patient interface of FIG. 2;

FIG. 4 is another embodiment of a tip compatible with the patient interface of FIG. 2;

FIG. 5 is an anterior view of the orbital septum and related structures of a medical patient;

FIG. 6 is a parasagittal sectional view of the orbital septum and related structures of the medical patient of FIG. 3; and

FIG. 7 is a flowchart illustrating one embodiment of a method of non-surgical orbital fat reduction that can be performed using the system of FIG. 1.

### DETAILED DESCRIPTION

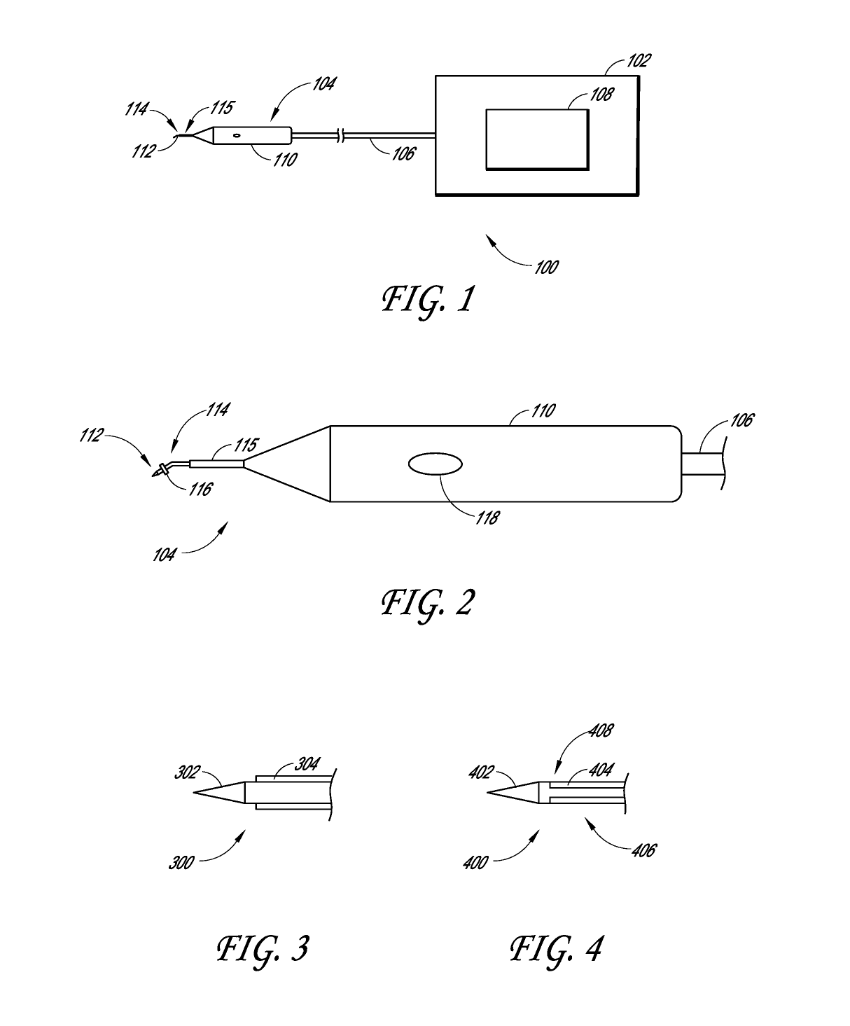

FIG. 1 illustrates a block diagram of one embodiment of a system configured to deliver electromagnetic energy to reduce the volume of orbital fat in a medical patient. The system 100 includes an energy source 102 and a patient interface 104. An energy conduit 106 couples the patient interface 104 to the energy source 102. The patient interface includes a handpiece portion 110 and a distal tip 112. The distal tip 112 is configured to be placed in contact with target tissue of the patient. Electromagnetic energy is generated by the energy source 102 and travels through the conduit 106 to the handpiece 110 and to the patient via the tip 112.

In some embodiments, the energy source 102 includes a radiofrequency (RF) generator. The source 102 can be configured to generate radiofrequency energy having a frequency in the megahertz (MHz) range. For example, in some embodiments, the source 102 generates RF energy having a frequency of 1, 2, 3, 4, 5, or greater than 5 MHz. In some embodiments, the source 102 generates RF energy having a frequency of about 1.4 MHz, 1.7 MHz, or between 1 and 2 MHz. The source 102 can be configured to deliver about 20, 30, 50, 100, 150, or 200 Watts of power. The actual power and/or frequency delivered by the source 102 can be controlled by the operator via the user interface 108.

In some embodiments, the shape of the energy waveform may be adjusted by the user. For example, in some embodiments, the energy source 102 generates a sinusoidal, partially rectified, or fully rectified energy waveform. In some embodiments, the energy source 102 generates a pulsed waveform. The waveform's duty cycle, or ratio of time on to period, may be adjusted as well. The wave form shape, pulse width, duty cycle, etc., can be controlled by the operator via the user interface 108.

Although many of the embodiments described herein relate to system that includes an RF energy source, in other embodiments, the energy source 102 provides a different form of energy in addition to or instead of RF energy. For example, the energy source 102 can include a laser, an intense, pulsed light source, electrical energy, and/or thermal energy source in addition to or instead of RF energy.

The conduit 106 connects to the energy source 102 at its proximal end via a detachable coupling so it may be remove and replaced, as desired. The conduit 106 connects to the proximal end of the patient interface 104 at the conduit's distal end. In some embodiments, the conduit 106 includes one, two, three, or more than three conductors that carry energy from the source 102 to the patient interface 104.

One embodiment of a patient interface 104 is shown in FIG. 2. The patient interface 104 includes a handpiece housing 110 and an electrode tip 112. In some embodiments, the electrode tip 112 is removably coupled to the handpiece housing 110. The electrode tip 112 can extend at least partially along a linear path, coaxially aligned with the handpiece housing 110. In some embodiments, the electrode tip 112 includes a bend 114 such that the distal portion of the tip 112 is oriented at a predetermined angle with respect to the longitudinal axis of the handpiece housing 110, such as shown in FIG. 2. The angle of the bend 114 between proximal and distal portions of the electrode tip 112 can be about 5, 10, 15, 30, 45, 60, or 90 degrees. In some embodiments, the tip 112 includes two bends 114 to form a Z- or S-shaped electrode.

The tip 112 is formed of a conductive material. For example, the tip 112 may be formed of a metal, such as stainless steel. In some embodiments, the tip is about 0.3 mm, 0.4 mm, 0.5 mm, 0.7 mm, 1.0 mm, 1.2 mm, 1.5 mm or about 2.0 mm in diameter. In some embodiments, the tip has a diameter less than about 0.5 mm. In some embodiments, the tip 112 has a diameter of about 0.005″, 0.007″ or 0.009″. In one embodiment, the tip 112 diameter is less than 0.010″. The tip 112 can be straight, bent, bendable, single-use, and/or disposable. A shaft adjacent the tip 112 can have a diameter of about 1.6 mm ( 1/16″) or about 2.4 mm ( 3/32″). In some embodiments, the shaft has a diameter less than 3.0 mm or less than 2.5 mm.

The length of the tip 112, measured from its end to the point where it couples to the shaft is sometimes about ¼″, ⅜″, ½″, ¾″, or 1″. In one embodiment, the patient interface 104 includes a bipolar electrode. In such embodiments, the handpiece housing 110 can be provided in the form of forceps, or tweezer, having two arms. A tip 112 may be provided at the distal end of each arm. In other embodiments, the patient interface 104 includes a unipole or monopole electrode

In one embodiment, as shown in FIG. 2, the patient interface 104 includes an optional stop 116 positioned near the distal end of the tip 112. The position of the stop 116 is adjustable by the user and allows the user to control the depth at which the distal end of the tip 112 penetrates the patient's tissue. In one embodiment, the stop 116 is threaded onto the tip 112. The position of the stop 116 is adjusted by rotating the stop 116 with respect to the tip 112. In another embodiment, the stop 116 is coupled to a sheath that is slidably mounted around the tip 112. The sheath can extend proximally to the handpiece 110. A slider or other control (not shown) coupled to the handpiece 110 can be used to extend or retract the sheath to change the position of the stop. In some embodiment, a control wire (e.g., a plastic tube or member) couples the stop 116 to a handpiece control. The control wire can extend along a side of the tip without surrounding or covering the tip 112. In some embodiments, the shaft position with respect to the tip 112 is adjustable and can operate as a stop 116.

In some embodiments, the entire distal portion of the tip 112 is a conductive electrode. In other embodiments, the tip 112 includes an insulating sleeve or layer applied to at least part of the tip's outside surface. Embodiments of such tips are illustrated in FIG. 3 and FIG. 4. The tip 300 of FIG. 3 includes a conductive portion 302 and an insulator 304. The conductive portion 302 is substantially cylindrical until it begins to taper towards its conical (or sometimes frustoconical), distal end. An insulator 304 surrounds the outside diameter of the cylindrical, conducive portion 302. The tip 400 of FIG. 4 also includes a cylindrical conductive portion 402 and an insulator 404. The cylindrical conductive portion 404 includes a recess 406. The insulator 404 is positioned within the recess 406 to provide a smooth, substantially continuous transition from the insulator 404 to the conductive portion 402, with no lip or rough edge that could catch on the patient's tissue. In yet another embodiment (not shown), the insulator 304 of the tip 300 of FIG. 3 tapers in thickness to provide a similar, smooth, substantially continuous transition from the insulator 304 to the conductive portion 302.

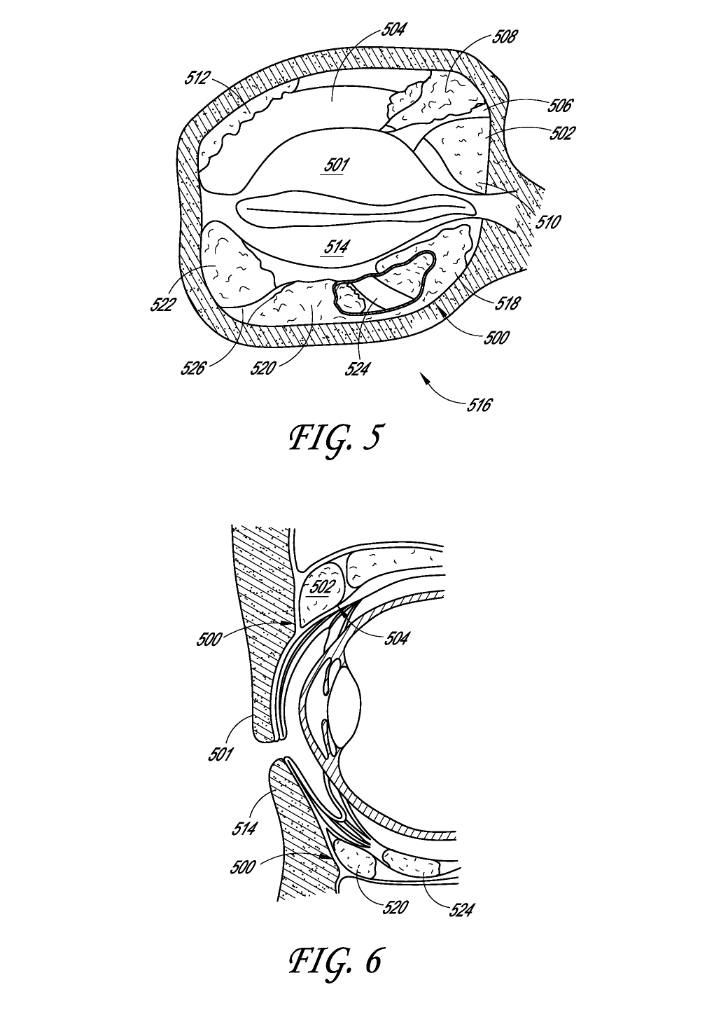

During use, the handpiece tip is inserted at least partially into a patient's fat pad. For example, the handpiece tip may be inserted into an ocular fat pad, as illustrated in FIG. 5 and FIG. 6. When provided with a stop, the depth of tip insertion may be controlled by setting the stop at an appropriate position along the tip's length. The handpiece is manipulated to point the tip in the desired direction. For example, the handpiece may be manipulated to point the tip away from the eyeball or other non-fat tissues within the patient's orbit. The user may activate the energy source to emit energy from the handpiece tip into the fat pad. As the energy is absorbed by the fat, the fat pad heats and shrinks in volume.

FIG. 5 provides an anterior view of the orbital septum 500 and related structures of a medical patient. FIG. 6 provides a parasagittal sectional view of the orbital septum and related structures. Upper eyelid 501 preaponeurotic fat 502 is located posterior to the orbital septum 500 and anterior to the levator aponeurosis 504. The trochlea 506 divides the preaponeurotic fat 502 into a central fat pad 508 and a medial fat pad 510. The lacrimal gland 512 resides within the lateral compartment. The medial fat pad 510 extends superomedial to the medial horn of the levator aponeurosis 504. A portion of the lateral end of the central fat pad 508 surrounds the medial aspect of the lacrimal gland 512. The lacrimal gland's 512 anterior border normally resides just behind the orbital margin, but involutional changes may lead to prolapse anteroinferiorly.

The lower eyelid 514 covers three retroseptal fat pads 516: the medial fat pad 518, the central fat pad 520, and the lateral fat pad 522. The medial and central fat pads 518, 520 are separated by the inferior oblique muscle 524. However, an isthmus of fat generally lies anterior to the muscle 524 belly.

The medial and lateral fat pads 518, 522 are separated by the arcuate expansion 526 of the inferior oblique 524, which extends from the capsulopalpebral fascia to the inferolateral orbital rim. The inferolateral orbital septum inserts about 2 mm outside the orbital rim, creating the recess of Eisner and allowing the lateral fat pad 522 to just spill over the orbital rim.

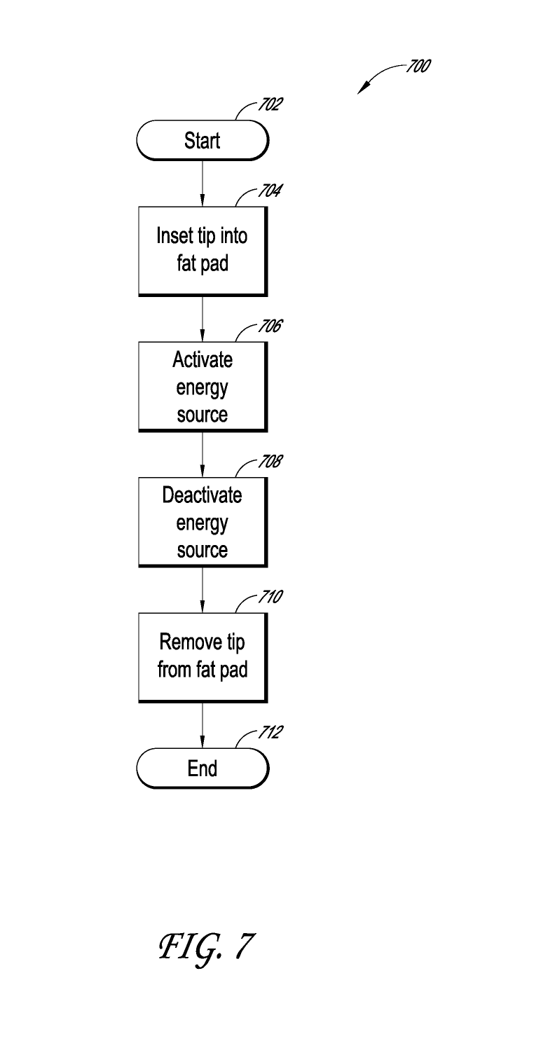

One embodiment of a method for non-surgical orbital fat reduction is illustrated in the flow chart of FIG. 7. The method 700 begins at block 702. At block 704 an energy conducting tip that is coupled to an electromagnetic energy source is inserted into a patient's fat pad. For example, the tip may be inserted into the fat pad through the patient's skin or through eyelid conjunctiva without penetrating the patient's skin. The tip and energy source can include any of the tips or energy sources described above. The fat pad can include an orbital fat, such as a preaponeurotic fat pad, a retroseptal fat pad, a central fat pad, a medial fat pad, and/or a lateral fat pad. In some embodiments, the fat pad includes a brow fat pad and/or a cheek fat pad.

At block 706, the energy source is activated. Energy from the energy source travels through the tip and into the fat pad. The energy can have any of the characteristics described herein. For example, the energy can have a user-selectable shape, power, energy intensity, duty cycle, etc. The absorbed energy heats the fat pad, which causes it to shrink in volume. After a desired treatment period has passed, the energy source is deactivated at block 708. The energy source may be deactivated by action of the user, such as by releasing a control (e.g., footswitch, button, control located on a handpiece, etc.). In other embodiments, the treatment period is programmed into the energy source such that the energy source automatically deactivates after a desired treatment period. In some embodiments, the treatment period is about 5, 10, 25, 50, 100, 200, or 500 ms. At block 710 the tip is removed from the patient's fat pad. The method 700 ends at block 712.

While the above detailed description has shown, described, and pointed out novel features as applied to various embodiments, it will be understood that various omissions, substitutions, and changes in the form and details of the devices or methods illustrated can be made without departing from the spirit of the disclosure. As will be recognized, certain embodiments of the inventions described herein can be embodied within a form that does not provide all of the features and benefits set forth herein, as some features can be used or practiced separately from others. The scope of certain inventions disclosed herein is indicated by the appended claims rather than by the foregoing description. All changes which come within the meaning and range of equivalency of the claims are to be embraced within their scope. Although certain embodiments and examples are disclosed above, inventive subject matter extends beyond the specifically disclosed embodiments to other alternative embodiments and/or uses and to modifications and equivalents thereof. Thus, the scope of the claims appended hereto is not limited by any of the particular embodiments described. For example, in any method or process disclosed herein, the acts or operations of the method or process can be performed in any suitable sequence and are not necessarily limited to any particular disclosed sequence.

Various operations can be described as multiple discrete operations in turn, in a manner that can be helpful in understanding certain embodiments; however, the order of description should not be construed to imply that these operations are order dependent. Additionally, the structures, systems, and/or devices described herein can be embodied as integrated components or as separate components. For purposes of comparing various embodiments, certain aspects and advantages of these embodiments are described. Not necessarily all such aspects or advantages are achieved by any particular embodiment. Thus, for example, various embodiments can be carried out in a manner that achieves or optimizes one advantage or group of advantages as taught herein without necessarily achieving other aspects or advantages as can also be taught or suggested herein. Thus, the invention is limited only by the claims that follow.

What is claimed is:

1. A method of nonsurgical orbital fat reduction, comprising:
providing an electromagnetic energy system, the electromagnetic energy system comprising an electromagnetic energy source and a patient interface coupled to the electromagnetic energy source, the patient interface comprising an elongate member configured to deliver electromagnetic energy generated by the electromagnetic energy source to tissue of a medical patient;
inserting at least a distal portion of the elongate member into an orbital fat pad of the medical patient; and
delivering a sufficient amount of electromagnetic energy from the electromagnetic energy source to the orbital fat pad to cause the orbital fat pad to shrink in volume.

2. The method of claim 1, wherein the orbital fat pad is selected from the group consisting of one or more of: a preaponeurotic fat pad, a retroseptal fat pad, a central fat pad, a medial fat pad, a lateral fat pad, and a brow fat pad.

3. The method of claim 1, further comprising orienting the elongate member such that the electromagnetic energy is not directed toward or delivered directly into tissue selected from the group consisting of one or more of: an eyeball, a trochlea, a lacrimal gland, a tendon and a muscle.

4. The method of claim 1, further comprising orienting the elongate member such that the electromagnetic energy is not substantially absorbed by tissue near the fat pad selected from the group consisting of one or more of: an eyeball, a trochlea, a lacrimal gland, a tendon and a muscle.

5. The method of claim 1, wherein the electromagnetic energy source is selected from the group consisting of one or more of: a radiofrequency generator, a laser, an intense pulsed light source, a thermal energy source, and an electrical energy source.

6. The method of claim 1, further comprising selecting at least one of a frequency, energy level, power level, waveform, and duty cycle of the electromagnetic energy's waveform.

7. The method of claim 6, wherein the frequency is selected to be about 1, 1.4, 1.7, 2, 3, 4, 5 MHz, or between 1 and 2 MHz.

8. The method of claim 6, wherein the power level is selected to be about 20, 30, 50, 100, 150, or 200 W.

9. The method of claim 1, further comprising adjusting a position of a stop coupled to the elongate member to control or limit a depth of insertion of the elongate member into the medical patient's orbital fat pad.

10. The method of claim 1, further comprising removing the elongate member from the orbital fat pad, inserting the elongate member into a different portion of the orbital fat pad, and delivering addition electromagnetic energy to the fat pad.

11. The method of claim 1, wherein said inserting at least the distal end of the elongate member comprises inserting at least the distal end of the elongate member through the medical patient's skin.

12. The method of claim 1, wherein said inserting at least the distal end of the elongate member comprises inserting at least the distal end of the elongate member through the medical patient's conjunctiva.

13. The method of claim 1, wherein said inserting at least the distal end of the elongate member comprises inserting at least the distal end of the elongate member into the fat pad without penetrating the medical patient's skin.

14. A method of nonsurgical orbital fat reduction, comprising:
penetrating a medical patient's tissue with a distal end of a radiofrequency (RF) probe;
advancing the distal end of the RF probe into an orbital fat pad; and
delivering RF energy from the RF probe into the orbital fat pad to shrink the orbital fat pad volume.

15. The method of claim 14, wherein the orbital fat pad is selected from the group consisting of one or more of: a preaponeurotic fat pad, a retroseptal fat pad, a central fat pad, a medial fat pad, a lateral fat pad, and a brow fat pad.

16. The method of claim 14, further comprising selecting at least one of a frequency, energy level, power level, waveform, and duty cycle of the RF energy waveform.

17. The method of claim 16, wherein the frequency is selected to be about 1 MHz, 1.4 MHz, 1.7 MHz, 2 MHz, 3 MHz, 4 MHz, 5 MHz, or between 1 and 2 MHz and the power level is selected to be about 20 W, 30 W, 50 W, 100 W, 150 W, or 200 W.

18. The method of claim 14, further comprising adjusting a position of a stop coupled to the RF probe to control or limit a depth of insertion of the RF probe into the medical patient's orbital fat pad.

19. The method of claim 1, wherein said advancing the distal end of the RF probe comprises inserting at least the distal end of the RF probe through the medical patient's conjunctiva.

20. The method of claim 1, wherein said inserting at least the distal end of the RF probe comprises inserting at least the distal end of the RF probe into the fat pad without penetrating the medical patient's skin.
